(19) United States
(12) Patent Application Publication
(10) Pub. No.: US 2026/0193224 A1
(43) Pub. Date: Jul. 9, 2026

(54) METHOD FOR PREPARING 5-HYDROXYMETHYLFURFURAL IN A TEMPERATURE-CONTROLLED PHASE SEPARATION SOLVENT SYSTEM

(71) Applicant: China Agricultural University, Beijing (CN)

(72) Inventors: Hongliang WANG, Beijing (CN); Fangyuan ZHOU, Beijing (CN); Wanbin ZHU, Beijing (CN)

(73) Assignee: China Agricultural University, Beijing (CN)

(21) Appl. No.: 19/537,574

(22) Filed: Feb. 12, 2026

Related U.S. Application Data
(63) Continuation of application No. PCT/CN2025/147658, filed on Dec. 30, 2025.

(30) Foreign Application Priority Data
Jan. 7, 2025 (CN) 2025100224439

(51) Int. Cl. C07D 307/50

(52) U.S. Cl. CPC C07D 307/50

(57) ABSTRACT

The present disclosure provides a method for preparing 5-hydroxymethylfurfural (HMF) in a temperature-controlled phase separation solvent system. The temperature-controlled phase separation solvent system is constructed using an aqueous betaine hydrochloride solution and an organic solvent. In this reaction system, carbohydrates can be converted into HMF without catalyst addition. During the heating reaction process, the aqueous betaine hydrochloride solution and the organic solvent form a biphasic system. After reaction, upon cooling at room temperature for a period of time, the betaine hydrochloride in the biphasic system precipitates in crystalline form. The present disclosure can not only catalyze the conversion of raw materials to obtain HMF with a high yield, but also greatly improve the recovery efficiency of reagents such as catalysts and solvents, thereby reducing the production cost of HMF. Meanwhile, the reaction system is environmentally friendly, has low requirements for equipment, and possesses the potential for large-scale industrial application.

### CROSS-REFERENCE TO RELATED APPLICATIONS

This application is a national stage application of PCT International Application No. PCT/CN2025/147658, filed on Dec. 30, 2025, which claims priority of Chinese Patent Application No. 202510022443.9, filed on Jan. 7, 2025. The contents of the prior applications are hereby incorporated by reference in their entirety.

### TECHNICAL FIELD

The present disclosure relates to the technical field of preparation of biomass-based high-value chemicals, and specifically to a method for efficiently preparing 5-hydroxymethylfurfural (HMF) in a temperature-controlled phase separation solvent system.

### BACKGROUND

The efficient and low-cost production of biomass-based chemicals is critical to developing a low-carbon, sustainable society. As a key biomass-derived platform compound, the industrial preparation of 5-hydroxymethylfurfural (HMF) has garnered substantial attention in recent years. HMF can be synthesized from carbohydrates, including cellulose, glucose, and fructose, via hydrolysis, isomerization, and dehydration reactions. The aldehyde group, hydroxymethyl group, and furan ring in its molecular structure confer unique physicochemical properties and reaction activity. HMF may be converted into a variety of high-value-added chemicals through diverse chemical processes, including 5-ethoxymethylfurfural (EMF), 2,5-dimethylfuran (DMF), 2,5-furandicarboxylic acid (FDCA), and levulinic acid (LA). These compounds exhibit significant application potential in fields such as biofuels, bioplastics, green solvents, and pharmaceuticals. Accordingly, HMF is recognized as a bridge connecting biomass refining and petrochemical refining, and has been designated by the U.S. Department of Energy as one of the “most promising biomass-based platform molecules”.

The aldehyde group, hydroxymethyl group, and furan ring in HMF enable it to derive numerous high-value products, but also render it highly unstable during preparation, separation, and purification processes, thereby making it prone to generating substantial by-products and ultimately resulting in low product yield and high cost. Developing and screening novel reaction solvents, as well as designing efficient catalytic systems, are key to enhancing the dehydration conversion of carbohydrates to HMF. In aqueous solutions, HMF is prone to rehydration into levulinic acid and formic acid, and to polymerization into humic substances and other by-products, thereby leading to low selectivity for the conversion of carbohydrates to HMF. Organic solvents have been proven to effectively improve HMF yield, among which the polar aprotic solvent dimethyl sulfoxide (DMSO) is widely used as a reaction medium for HMF production from carbohydrates. In the absence of external catalysts, fructose can be efficiently converted to HMF in DMSO through solvation effects, with a yield of up to 77% (ChemSusChem, 2019, 12(10): 2211-2219). However, separating HMF from these solvents requires considerable energy consumption, thereby hindering their commercial application. Ionic liquids and deep eutectic solvent systems exhibit good solubility for carbohydrates and can suppress the further hydrolysis of HMF. Nevertheless, the recycling and reuse of these solvents remain incompletely resolved, leading to high HMF production costs. Currently, there are few industrial-scale production examples.

In addition, biphasic systems, which incorporate an organic extraction phase during the reaction process, enable the continuous extraction of the formed HMF from the reaction phase to the organic phase. This can inhibit side reactions such as rehydration, decomposition, and polymerization of HMF to a certain extent, thereby enhancing the yield of HMF. However, conventional biphasic systems generally suffer from problems such as low extraction efficiency (a poor distribution ratio of HMF) and difficulty in recovering auxiliaries and catalysts, which severely restrict their industrial application.

Based on the foregoing, the present disclosure is hereby provided.

### SUMMARY

In view of the technical problems or shortcomings in the aforementioned prior art, the present disclosure provides a method for efficiently preparing 5-hydroxymethylfurfural (HMF) in a temperature-controlled phase separation solvent system. By introducing a temperature-controlled phase change additive (betaine hydrochloride in the present disclosure) into the reaction system, the present disclosure effectively resolves issues including substance separation, recovery, and product purification in biphasic systems. At room temperature, the temperature-controlled phase-change additive is insoluble in the reaction system, thereby facilitating its separation from the system and reducing the recovery costs of the additive and catalyst. At the reaction temperature, the temperature-controlled phase-change additive dissolves in the reaction phase and promotes phase separation between the reaction phase and the extraction phase, enabling in-situ extraction of HMF into the organic phase with a high distribution ratio, and thus significantly enhancing the yield of HMF.

To achieve the above objectives, the technical scheme of the present disclosure is as follows:

The present disclosure provides a method for preparing 5-hydroxymethylfurfural (HMF) in a temperature-controlled phase separation solvent system, which comprises the following steps:
S1: Constructing a temperature-controlled phase separation solvent system; wherein the solvent system comprises an aqueous betaine hydrochloride solution and an organic solvent;
S2: Adding a defined amount of carbohydrate to the solvent system and heating the mixture to conduct a dehydration reaction, with the aqueous betaine hydrochloride solution serving as the reaction phase and the organic solvent serving as the extraction phase to form phase stratification;
S3: After completion of the reaction, cooling the reaction mixture at room temperature for a period of time, whereby betaine hydrochloride precipitates in crystalline form; the product is present in a mixed solution of the organic phase and deionized water, and 5-hydroxymethylfurfural is obtained via isolation and purification of the mixed solution.

Further, in step S1, a temperature-controlled phase separation solvent system is constructed with varying volume ratios of the organic solvent to the aqueous betaine hydrochloride solution. The volume ratio of the organic solvent to the aqueous betaine hydrochloride solution ranges from 0.5:1 to 8:1. Preferably, in step S1, the volume ratio of the organic solvent to the aqueous betaine hydrochloride solution is 1:1, 2:1, 3:1, 4:1, or 5:1, with 4:1 being more preferred.

Further, in step S1, the concentration of betaine hydrochloride in the aqueous betaine hydrochloride solution ranges from 5 to 50 wt %. Preferably, the concentration is 40 wt %.

Further, in step S1, the organic solvent is one or more selected from acetone, ethyl acetate, tetrahydrofuran, 2-methyltetrahydrofuran, and acetonitrile. Preferably, the organic solvent is acetone.

Further, in step S2, the concentration of the carbohydrate refers to its concentration in the aqueous betaine hydrochloride solution. Preferably, the concentration of the carbohydrate is 5 wt %, 10 wt %, 20 wt %, 30 wt %, 40 wt %, 50 wt %, or 60 wt %. More preferably, the carbohydrate concentration does not exceed 30 wt %.

Further, in step S2, the carbohydrate is any one selected from fructose, high-fructose syrup, sucrose, and inulin.

Further, in step S2, the reaction temperature ranges from 60 to 180° C., preferably from 90 to 120° C.

Further, in step S2, the reaction time ranges from 5 to 360 minutes, preferably from 10 to 240 minutes.

Further, in step S3, after the reaction is completed, a separation and purification step is further included:
immediately cooling the reaction mixture to room temperature and standing at room temperature for a period of time until most of the betaine hydrochloride crystallizes and precipitates out;
separating and collecting the betaine hydrochloride and the organic phase, respectively;
distilling the organic phase to obtain the 5-hydroxymethylfurfural; and the recovered organic solvent and betaine hydrochloride are directly recycled for subsequent use.

Further, an acidic catalyst may be additionally added to the reaction system of the present disclosure to accelerate the reaction. The acidic catalyst is one or more selected from inorganic acids, organic acids, acidic ion exchange resins, and acidic metal salt compounds. Preferably, the mass of the acidic catalyst is from 0.05 to 10% of the total mass of the sugar raw material.

Preferably, the inorganic acid includes one or more of sulfuric acid, hydrochloric acid, and phosphoric acid; the organic acid includes one or more of sulfonic acid, trichloroacetic acid, and acetic acid; the acidic ion exchange resin includes one or more of sulfonic acid-based cation exchange resins and carboxylic acid-based cation exchange resins; the acidic metal salt compound includes one or more of SnCl₄, AlCl₃, CrCl₃, and ZnCl₂.

Compared with the prior art, the advantages and positive effects of the present disclosure are as follows:
(1) The present disclosure develops a temperature-controlled phase separation solvent system that is constructed from an aqueous betaine hydrochloride solution and an organic solvent. During the heating reaction process, the aqueous betaine hydrochloride solution and the organic solvent form a biphasic system. After the reaction, upon cooling at room temperature for a period of time, the betaine hydrochloride in the biphasic system precipitates in crystalline form, thereby facilitating the recovery and recycling of both betaine hydrochloride and the organic solvent. The one-pot preparation of 5-hydroxymethylfurfural (HMF) in this reaction system features a simple process, minimal by-products (e.g., formic acid and levulinic acid), high selectivity, and a yield of 5-hydroxymethylfurfural exceeding 90%, which enables the efficient conversion of carbohydrates.
(2) The solvent system of the present disclosure requires no additional catalyst, as it inherently possesses acid-catalytic functionality. Compared to conventional homogeneous acid catalysts (e.g., sulfuric acid, hydrochloric acid, phosphoric acid, or metal salts), the system reduces the generation of by-products, the corrosion of equipment, and the discharge of environmentally harmful waste liquids, demonstrating greater potential for industrial application.

### BRIEF DESCRIPTION OF DRAWINGS

To more clearly illustrate the technical solutions described in the embodiments of the present disclosure or in the prior art, a brief description of the accompanying drawings employed in the description of the embodiments or prior art is provided below. It is apparent that the accompanying drawings in the following description merely illustrate some embodiments of the present disclosure, and for those skilled in the art, other accompanying drawings may also be obtained based on these accompanying drawings without exerting creative efforts.

FIG. 1 is a schematic diagram of the overall reaction process for preparing 5-hydroxymethylfurfural in the temperature-controlled phase separation solvent system;

FIG. 2 is a schematic diagram of the fructose dehydration process in the betaine hydrochloride/acetone reaction system;

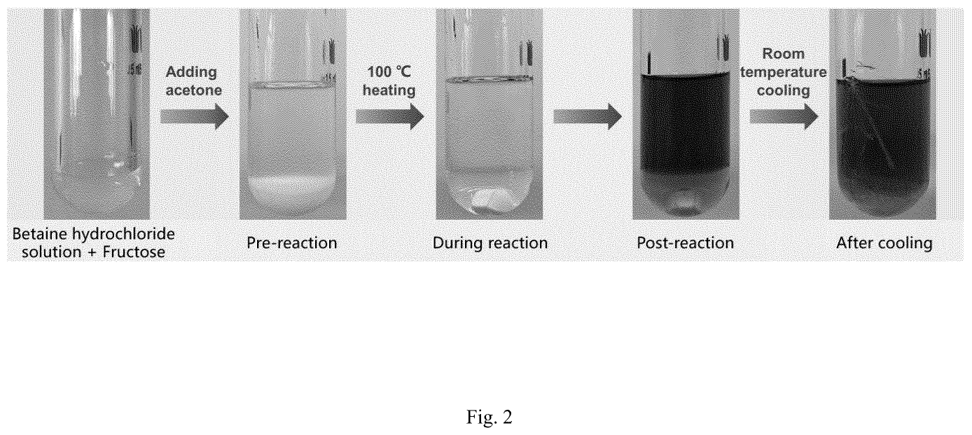

FIG. 3 is a comparative graph showing the impact of acetone/betaine hydrochloride solution systems with different volume ratios on the recovery rate of betaine hydrochloride.

### DETAILED DESCRIPTION OF THE EMBODIMENTS

The present disclosure provides a method for preparing 5-hydroxymethylfurfural (HMF) in a temperature-controlled phase separation solvent system. The temperature-controlled phase separation solvent system is constructed using an aqueous betaine hydrochloride solution and an organic solvent. In this reaction system, carbohydrates can be converted into HMF without catalyst addition, though the inclusion of an additional catalyst can shorten the reaction time. During the heating reaction process, the aqueous betaine hydrochloride solution and the organic solvent form a biphasic system. After the reaction, upon cooling at room temperature for a period of time, the betaine hydrochloride in the biphasic system precipitates in crystalline form. Carbohydrates are efficiently converted to HMF in this solvent system with reduced side reactions. This system also enables the recycling of both betaine hydrochloride and the organic solvent.

The method for preparing 5-hydroxymethylfurfural in a temperature-controlled phase separation solvent system of the present disclosure includes the following steps:
S1: Constructing a carbohydrate dehydration reaction system, which includes a carbohydrate raw material, betaine hydrochloride, deionized water, and an organic extraction solvent. At room temperature, after adding the extraction solvent, the betaine hydrochloride is insoluble in the aqueous carbohydrate solution, while the extraction solvent is miscible with the aqueous carbohydrate solution.
S2: Heating to carry out a dehydration reaction. Upon heating, the betaine hydrochloride dissolves into the reaction solution to form a reaction phase, while the extraction solvent separates from the reaction solution to form an organic phase. The generated HMF can be timely extracted into the organic phase.
S3: After the reaction is completed, cooling the reaction mixture to reduce the temperature, whereby the betaine hydrochloride precipitates out from the reaction system.
S4: Performing solid-liquid separation to recover betaine hydrochloride. The recovered betaine hydrochloride can be directly reused in subsequent batches.
S5: Distilling the extraction solvent and deionized water to separate 5-hydroxymethylfurfural. The extraction solvent and water are recycled for subsequent use.

The betaine hydrochloride adopted in the present disclosure functions both as a catalyst for saccharide dehydration and as a temperature-controlled phase separation additive. At elevated temperatures, it can promote phase separation between the reaction solution and the extraction solvent to form a biphasic system. At room temperature, it can automatically crystallize and precipitate to facilitate separation and recovery. This system can not only catalyze the conversion of raw materials such as high-concentration fructose (>30 wt %) to obtain 5-hydroxymethylfurfural with a high yield (>85%), but also greatly improve the recovery efficiency of reagents such as catalysts and solvents, thereby reducing the production cost of 5-hydroxymethylfurfural. Meanwhile, the reaction system is environmentally friendly, has low requirements for equipment, and possesses the potential for large-scale industrial application.

The present disclosure will be further described below in conjunction with specific examples to enable those skilled in the art to better understand the present disclosure. However, the present disclosure is not limited to the following examples. It should be noted that all modifications and equivalent implementations obviously made to the embodiments or examples of the present disclosure by those skilled in the art without exerting creative efforts shall fall within the scope of protection defined by the appended claims.

Unless otherwise specified, all reagents and materials used in the following examples are commercially available. For example, fructose used in the examples below of the present disclosure has a chemical formula of C₆H₁₂O₆, a molecular weight of 180.15, and a CAS number of 7660-25-5. Betaine hydrochloride used in the examples below has a chemical formula of C₅H₁₂ClNO₂, a molecular weight of 153.61, and a CAS number of 590-46-5.

In the following examples of the present disclosure, 5-hydroxymethylfurfural (HMF) is detected by high-performance liquid chromatography (HPLC).

The specific test conditions are as follows: the mobile phase used for the test is a 5 mM dilute sulfuric acid solution, the flow rate is 0.6 mL/min, the chromatographic column is HPX-87H, the test temperature is 40° C., and the injection volume is 10 μL.

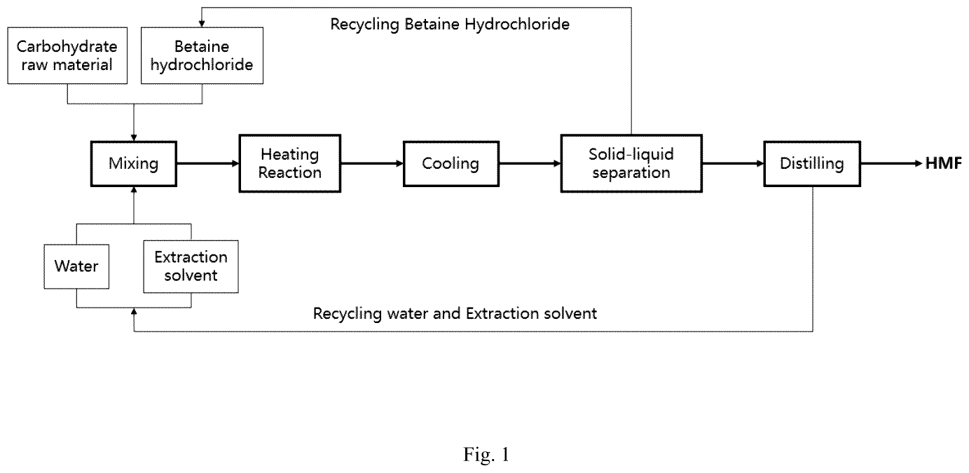

The overall reaction process for preparing HMF in the controllable phase separation solvent system of the present disclosure is illustrated in FIG. 1. A certain amount of carbohydrate is dissolved in an aqueous betaine hydrochloride solution; upon addition of acetone, a portion of betaine hydrochloride precipitates out. During the heating reaction process, the precipitated betaine hydrochloride redissolves, and the aqueous betaine hydrochloride solution and acetone form a biphasic system. The carbohydrate undergoes dehydration in the aqueous betaine hydrochloride solution to generate HMF, which is concurrently in-situ extracted into the acetone phase, thus reducing the formation of by-products. After the reaction is completed, the reaction mixture is immediately cooled to room temperature and allowed to stand for a period of time, during which most of the betaine hydrochloride precipitates out from the solution system in crystalline form, allowing for easy separation and reuse. Subsequently, an excess amount of acetone is added to fully precipitate betaine hydrochloride; acetone and water are then removed by reduced-pressure distillation to yield HMF.

### Example 1: Investigation on the Impact of Solvent Systems Constructed from Different Organic Solvents and Aqueous Betaine Hydrochloride Solution on the Conversion of Fructose to HMF

0.22 g of fructose and 2 mL of 40 wt % aqueous betaine hydrochloride solution were added to a 15 mL thick-walled pressure-resistant bottle, and fructose was dissolved via magnetic stirring. Then, 6 mL of an organic solvent (selected from acetone, ethyl acetate, tetrahydrofuran, 2-methyltetrahydrofuran, or acetonitrile) was added and mixed uniformly. The hydrothermal reaction was carried out in an oil bath at 100° C. for 120 minutes with a stirring speed of 800 rpm. After the reaction was completed, the reaction mixture was immediately cooled to room temperature and allowed to stand at room temperature for 2 hours, during which betaine hydrochloride crystallized and precipitated out. The solid was separated, and the acetone organic phase was collected separately. After dilution with deionized water, the fructose conversion rate and the yield of 5-hydroxymethylfurfural (HMF) were determined by high-performance liquid chromatography (HPLC). According to the experimental results, compared with other organic solvents, the system composed of acetone and the aqueous betaine hydrochloride solution exhibited superior fructose conversion rate and HMF yield. Therefore, all subsequent examples were carried out in the acetone/betaine hydrochloride system.

$$Fructose concersion rate = 1 - \frac{\begin{matrix}Mass of residual fructose in the \\ reaction system after the reaction\end{matrix}}{Initial mass of fructose added to the reaction system} \times 100 \%$$

$$HMF yield = 1 - \frac{Molar amount of HMF generated after the reaction}{Molar amount of fructose input before the reaction} \times 100 \%$$

### Example 2: Investigation on the Impact of Solvent Systems Constructed from Aqueous Betaine Hydrochloride Solutions with Different Concentrations and Acetone on the Conversion of Fructose to HMF

Aqueous solutions of betaine hydrochloride with different concentrations were prepared. A specified amount of betaine hydrochloride was added to a beaker according to the target concentration, followed by the addition of a certain amount of deionized water. The mixture was stirred in a water bath at 50° C. until the liquid became clear and free of solids, yielding aqueous betaine hydrochloride solutions with concentrations of 5 wt %, 10 wt %, 15 wt %, 20 wt %, 25 wt %, 30 wt %, 35 wt %, 40 wt %, 45 wt % and 50 wt %, respectively. Finally, the prepared aqueous solutions of betaine hydrochloride were cooled to room temperature and stored in volumetric flasks to prevent water evaporation.

0.22 g of fructose and 2 mL of the aqueous betaine hydrochloride solution (with a concentration of 5 wt %, 10 wt %, 15 wt %, 20 wt %, 25 wt %, 30 wt %, 35 wt %, 40 wt %, 45 wt % or 50 wt %) were added to a 15 mL thick-walled pressure-resistant bottle, and fructose was dissolved via magnetic stirring. Then, 6 mL of acetone was added and mixed, followed by a hydrothermal reaction carried out in an oil bath at 100° C. for 120 minutes with a stirring speed of 800 rpm. After the reaction was completed, the reaction mixture was immediately cooled to room temperature and allowed to stand at room temperature for 2 hours, during which betaine hydrochloride crystallized and precipitated out. The solid was separated, and the acetone organic phase was collected separately. After dilution with deionized water, the fructose conversion rate and 5-hydroxymethylfurfural (HMF) yield were determined by high-performance liquid chromatography (HPLC).

As shown in Table 1, under the same reaction conditions, the fructose conversion rate increased with the increase in the concentration of betaine hydrochloride. The HMF yield reached a maximum of 86.57% at a betaine hydrochloride concentration of 40 wt %, and then decreased with a further increase in betaine hydrochloride concentration. This was mainly because the increase in the betaine hydrochloride concentration elevated the acidity of the reaction system, thereby improving the fructose conversion rate. An appropriate betaine hydrochloride concentration of 40 wt % was thus selected for subsequent reactions. It should be noted that a betaine hydrochloride concentration of at least 20 wt % was required to form a biphasic system with acetone under heating conditions.

TABLE 1
Effect of Aqueous betaine hydrochloride solution with Different
Concentrations on the Conversion of Fructose to HMF

| | | Concentration | | | | | |
|---|---|---|---|---|---|---|---|
| | | of Aqueous | Volume of | | | | |
| | | Betaine | Aqueous | | | | |
| | Fructose | Hydrochloride | Betaine | Acetone | Reaction | Fructose | HMF |
| Sample | Dosage | Solution | Hydrochloride | Volume | Temperature/ | Conversion | Yield |
| No. | (g) | (wt %) | Solution (mL) | (mL) | Time | Rate (%) | (%) |
| 1 | 0.22 g | 0 | 2 | 6 | 100° C., 120 min | 0.00 | 0.00 |
| 2 | 0.22 g | 5 | 2 | 6 | 100° C., 120 min | 20.48 | 49.20 |
| 3 | 0.22 g | 10 | 2 | 6 | 100° C., 120 min | 34.56 | 69.61 |
| 4 | 0.22 g | 15 | 2 | 6 | 100° C., 120 min | 48.78 | 75.67 |
| 5 | 0.22 g | 20 | 2 | 6 | 100° C., 120 min | 60.97 | 76.36 |
| 6 | 0.22 g | 25 | 2 | 6 | 100° C., 120 min | 74.53 | 80.40 |
| 7 | 0.22 g | 30 | 2 | 6 | 100° C., 120 min | 86.58 | 80.90 |
| 8 | 0.22 g | 35 | 2 | 6 | 100° C., 120 min | 93.57 | 83.36 |
| 9 | 0.22 g | 40 | 2 | 6 | 100° C., 120 min | 97.14 | 86.57 |
| 10 | 0.22 g | 45 | 2 | 6 | 100° C., 120 min | 98.62 | 84.32 |
| 11 | 0.22 g | 50 | 2 | 6 | 100° C., 120 min | 99.55 | 83.90 |

### Example 3: Investigation on the Impact of Volume Ratio of Organic Solvent to Aqueous Betaine Hydrochloride Solution on the Conversion of Fructose to HMF

0.22 g of fructose and 2 mL of 40 wt % aqueous betaine hydrochloride solution were added to a 15 mL thick-walled pressure-resistant bottle, and fructose was dissolved via magnetic stirring. Then, different volumes of acetone (0 mL, 2 mL, 4 mL, 6 mL, 8 mL or 10 mL) were added and mixed, followed by a hydrothermal reaction carried out in an oil bath at 100° C. for 120 minutes with a stirring speed of 800 rpm. After the reaction was completed, the reaction mixture was immediately cooled to room temperature and allowed to stand at room temperature for 2 hours, during which betaine hydrochloride crystallized and precipitated out. The solid was separated, and the remaining mixture was diluted with deionized water. The fructose conversion rate and HMF yield were determined by HPLC.

As shown in Table 2, with a fixed volume of 2 mL aqueous betaine hydrochloride solution, both the fructose conversion rate and HMF yield increased with the increase in the volume of added acetone. A high HMF yield of 89.44% was achieved when the volume ratio of acetone to aqueous betaine hydrochloride solution reached 8:2. Further increasing acetone dosage resulted in only a marginal improvement in HMF yield. Therefore, the solvent system with an acetone-to-aqueous betaine hydrochloride solution volume ratio of 8:2 was selected for subsequent examples.

TABLE 2
Effect of Acetone Dosage on the Conversion of Fructose to HMF

| | | Volume of 40 wt % | | | | |
|---|---|---|---|---|---|---|
| | Fructose | Aqueous Betaine | Acetone | Reaction | Fructose | HMF |
| Sample | Dosage | Hydrochloride | Volume | Temperature/ | Conversion | Yield |
| No. | (g) | Solution (mL) | (mL) | Time | Rate (%) | (%) |
| 1 | 0.22 g | 2 | 0 | 100° C., 120 min | 78.38 | 51.42 |
| 2 | 0.22 g | 2 | 2 | 100° C., 120 min | 86.88 | 68.82 |
| 3 | 0.22 g | 2 | 4 | 100° C., 120 min | 93.03 | 78.36 |
| 4 | 0.22 g | 2 | 6 | 100° C., 120 min | 97.14 | 84.10 |
| 5 | 0.22 g | 2 | 8 | 100° C., 120 min | 98.71 | 89.44 |
| 6 | 0.22 g | 2 | 10 | 100° C., 120 min | 98.95 | 89.76 |

### Example 4: Investigation on the Impact of Substrate Concentration on the Conversion of Fructose to HMF

A defined amount of fructose (0.11 g, 0.22 g, 0.50 g, 0.86 g, 1.33 g, 2.00 g or 3.00 g) and 2 mL of 40 wt % aqueous betaine hydrochloride solution were added to a 15 mL thick-walled pressure-resistant bottle, and fructose was dissolved via magnetic stirring to form fructose solutions with different concentrations (5 wt %, 10 wt %, 20 wt %, 30 wt %, 40 wt %, 50 wt % or 60 wt %). Then, 8 mL of acetone was added and mixed, followed by a hydrothermal reaction carried out in an oil bath at 100° C. for 120 minutes with a stirring speed of 800 rpm. After the reaction was completed, the reaction mixture was immediately cooled to room temperature and allowed to stand at room temperature for 2 hours, during which betaine hydrochloride crystallized and precipitated out. The solid was separated, and the remaining mixture was diluted with deionized water. The fructose conversion rate and HMF yield were determined by HPLC.

As shown in Table 3, under the same reaction conditions, both the fructose conversion rate and HMF yield decreased with the increase in fructose concentration. The HMF yield remained above 80% when the fructose concentration was 30 wt %, whereas the yield dropped to 56.97% when the fructose concentration was increased to 60 wt %. This phenomenon is primarily attributed to incomplete conversion of a portion of fructose, and the enhanced formation of by-products at elevated fructose concentrations.

TABLE 3
Effect of Substrate Concentration on the Conversion of Fructose to HMF

| | | Volume of 40 wt % | | | | |
|---|---|---|---|---|---|---|
| | Fructose | Aqueous Betaine | Acetone | Reaction | Fructose | HMF |
| Sample | Concentration | Hydrochloride | Volume | Temperature/ | Conversion | Yield |
| No. | (wt %) | Solution (mL) | (mL) | Time | Rate (%) | (%) |
| 1 | 5 | 2 | 8 | 100° C., 120 min | 99.30 | 92.64 |
| 2 | 10 | 2 | 8 | 100° C., 120 min | 98.71 | 89.44 |
| 3 | 20 | 2 | 8 | 100° C., 120 min | 97.13 | 85.72 |
| 4 | 30 | 2 | 8 | 100° C., 120 min | 94.29 | 81.89 |
| 5 | 40 | 2 | 8 | 100° C., 120 min | 89.18 | 73.67 |
| 6 | 50 | 2 | 8 | 100° C., 120 min | 85.43 | 67.38 |
| 7 | 60 | 2 | 8 | 100° C., 120 min | 79.85 | 56.97 |

### Example 5: Investigation on the Impact of Reaction Temperature and Time on the Conversion of Fructose to HMF

0.86 g of fructose and 2 mL of 40 wt % aqueous betaine hydrochloride solution were added to a 15 mL thick-walled pressure-resistant bottle, and fructose was dissolved via magnetic stirring to form an aqueous fructose solution with a concentration of 30 wt %. Then, 8 mL of acetone was added and mixed, followed by a hydrothermal reaction carried out in an oil bath at a stirring speed of 800 rpm, with the heating temperature ranging from 90° C. to 120° C. and the reaction time ranging from 10 min to 240 min. After the reaction was completed, the reaction mixture was immediately cooled to room temperature and allowed to stand at room temperature for 2 hours, during which betaine hydrochloride crystallized and precipitated out. The solid was separated, and the remaining mixture was diluted with deionized water. The fructose conversion rate and HMF yield were determined by HPLC.

As shown in Table 4, increasing the reaction temperature accelerated both the fructose conversion rate and the HMF formation rate. The fructose solution with a concentration of 30 wt % achieved a relatively high HMF yield over the temperature range of 90° C. to 120° C. and the reaction time range of 10 min to 240 min. Specifically, the HMF yield reached 85.63% when reacted at 100° C. for 180 min, 85.69% at 110° C. for 90 min, and 86.55% at 120° C. for 40 min.

TABLE 4
Effect of Reaction Temperature and Reaction
Time on the Conversion of Fructose to HMF

| | | Volume of 40 wt % | | | | | |
|---|---|---|---|---|---|---|---|
| | Fructose | Aqueous Betaine | Acetone | Reaction | | Fructose | HMF |
| Sample | Dosage | Hydrochloride | Volume | Temperature | Time | Conversion | Yield |
| No. | (g) | Solution (mL) | (mL) | (° C.) | (min) | Rate (%) | (%) |
| 1 | 0.86 | 2 | 8 | 90 | 30 | 47.31 | 17.66 |
| 2 | 0.86 | 2 | 8 | 90 | 60 | 64.84 | 34.19 |
| 3 | 0.86 | 2 | 8 | 90 | 120 | 75.20 | 49.25 |
| 4 | 0.86 | 2 | 8 | 90 | 180 | 83.12 | 62.15 |
| 5 | 0.86 | 2 | 8 | 90 | 240 | 89.05 | 71.06 |
| 6 | 0.86 | 2 | 8 | 100 | 60 | 79.36 | 59.35 |
| 7 | 0.86 | 2 | 8 | 100 | 90 | 88.91 | 74.73 |
| 8 | 0.86 | 2 | 8 | 100 | 120 | 94.29 | 81.89 |
| 9 | 0.86 | 2 | 8 | 100 | 180 | 98.88 | 85.63 |
| 10 | 0.86 | 2 | 8 | 110 | 15 | 70.08 | 43.71 |
| 11 | 0.86 | 2 | 8 | 110 | 30 | 86.67 | 68.98 |
| 12 | 0.86 | 2 | 8 | 110 | 60 | 96.45 | 83.00 |
| 13 | 0.86 | 2 | 8 | 110 | 90 | 99.58 | 85.69 |
| 14 | 0.86 | 2 | 8 | 120 | 10 | 78.87 | 57.21 |
| 15 | 0.86 | 2 | 8 | 120 | 20 | 95.92 | 81.88 |
| 16 | 0.86 | 2 | 8 | 120 | 30 | 98.80 | 85.34 |
| 17 | 0.86 | 2 | 8 | 120 | 40 | 100.00 | 86.55 |

### Example 6: Investigation on the Impact of Acetone Dosage on the Recovery Rate of Betaine Hydrochloride

2 mL of 40 wt % aqueous betaine hydrochloride solution was added to a 15 mL thick-walled pressure-resistant bottle, followed by the addition of different volumes of acetone and thorough mixing to form acetone/aqueous betaine hydrochloride solution systems with various volume ratios of acetone to the aqueous betaine hydrochloride solution (0:1, 1:1, 2:1, 3:1, 4:1, 5:1, 6:1, 7:1, or 8:1). Subsequently, the mixture was heated in an oil bath at 100° C. for 5 min with a stirring speed of 800 rpm until the partially precipitated betaine hydrochloride redissolved. Finally, the reaction mixture was immediately cooled to room temperature and allowed to stand at room temperature for 2 hours, during which betaine hydrochloride crystallized and precipitated out. The solid was separated and dried in a vacuum drying oven at 80° C. for 12 hours. After drying, the sample was weighed, and the recovery rate of betaine hydrochloride was calculated accordingly.

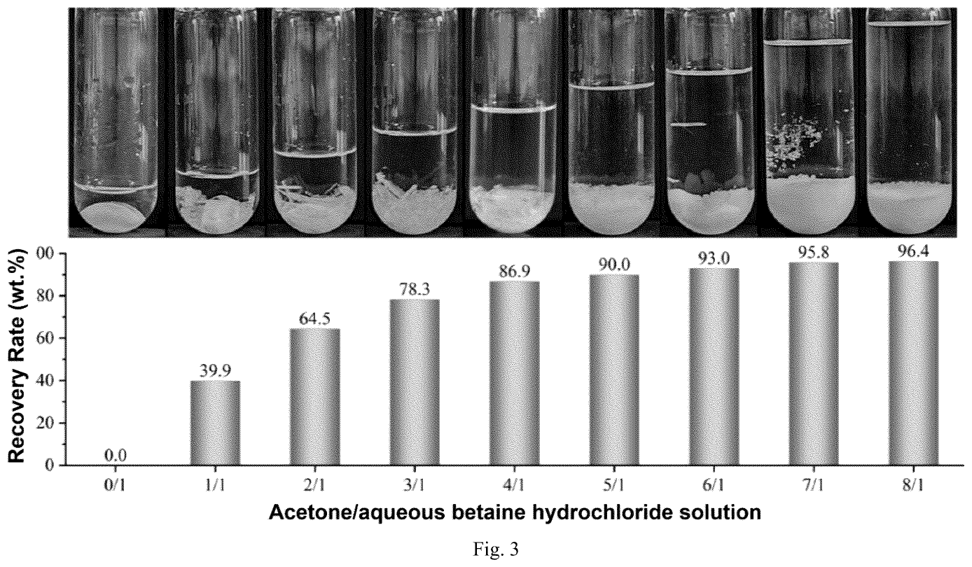

As shown in FIG. 3, increasing the acetone dosage to elevate the volume ratio of acetone to aqueous betaine hydrochloride solution could significantly improve the recovery rate of betaine hydrochloride. In the absence of acetone, no betaine hydrochloride crystals precipitated out when the aqueous betaine hydrochloride solution was left to stand at room temperature. When the volume ratio of acetone to aqueous betaine hydrochloride solution was 8:1, the recovery rate of betaine hydrochloride reached as high as 96.4%. These results indicate that the recovery of betaine hydrochloride can be achieved by increasing the acetone dosage, thus enabling the cyclic utilization of the solvent system, which holds significant industrial application value.

### Example 7: Conversion of Different Carbohydrates to HMF in an Acetone/Betaine Hydrochloride Solvent System

0.29 g or 1.15 g of high-fructose syrup (sugar content: 5 wt % glucose and 70 wt % fructose), 0.8 g of betaine hydrochloride and a specified amount of deionized water were added to a 15 mL thick-walled pressure-resistant bottle, and the mixture was dissolved with magnetic stirring to form sugar solutions with concentrations of 10 wt % or 30 wt %. Then, 8 mL of acetone was added and mixed uniformly, followed by a hydrothermal reaction carried out in an oil bath at 120° C. for 30 min with a stirring speed of 800 rpm. After the reaction was completed, the reaction mixture was immediately cooled to room temperature and allowed to stand at room temperature for 2 hours, during which betaine hydrochloride crystallized and precipitated out. The solid was separated, and the remaining mixture was diluted with deionized water. The fructose conversion rate and HMF yield were determined by HPLC.

0.22 g of sucrose or 0.22 g of inulin (CAS No. 9005-80-5) and 2 mL of 40 wt % aqueous betaine hydrochloride solution were added to a 15 mL thick-walled pressure-resistant bottle, and the mixture was dissolved with magnetic stirring to form a sugar solution with a concentration of 10 wt %. Then, 8 mL of acetone was added and mixed, followed by a hydrothermal reaction carried out in an oil bath at 120° C. for 30 min with a stirring speed of 800 rpm. After the reaction was completed, the reaction mixture was immediately cooled to room temperature and allowed to stand at room temperature for 2 hours, during which betaine hydrochloride crystallized and precipitated out. The solid was separated, and the remaining mixture was diluted with deionized water. The fructose conversion rate and HMF yield were determined by HPLC.

As shown in Table 5, under the same reaction conditions, the HMF yield followed the descending order: high-fructose syrup>inulin>sucrose. Notably, after the reaction of high-fructose syrup and sucrose, a large portion of glucose remained unconverted in the reaction system. This indicates that the reaction system can selectively promote the conversion of fructose to HMF while retaining most of the glucose, which can be recovered for further utilization.

TABLE 5
Conversion of Different Carbohydrates to HMF in
the Acetone/Betaine Hydrochloride Solvent System

| | Volume | | | | |
|---|---|---|---|---|---|
| | Ratio | | | Sugar | |
| | of Betaine | Sugar | | Con- | |
| | Hydrochloride | Type/Concen- | Reaction | version | HMF |
| Sample | Solution to | tration | Temper- | Rate | Yield |
| No. | Acetone | (wt %) | ature/Time | (%) | (%) |
| 1 | 1/4 | High-fructose | 120° C., | 93.15 | 84.56 |
| | | syrup/10 | 30 min | | |
| 2 | 1/4 | High-fructose | 120° C., | 93.10 | 79.37 |
| | | syrup/30 | 30 min | | |
| 3 | 1/4 | Sucrose/10 | 120° C., | 100 | 47.68 |
| | | | 30 min | | |
| 4 | 1/4 | Inulin/10 | 120° C., | 86.85 | 79.47 |
| | | | 30 min | | |

### Example 8: Investigation on the Impact of Additional Acidic Catalyst on the Conversion of Carbohydrate to HMF

0.22 g of fructose and 2 mL of 40 wt % aqueous betaine hydrochloride solution were added to a 15 mL thick-walled pressure-resistant bottle and fructose was dissolved via magnetic stirring. Then, 8 mL of acetone was added and mixed. Finally, an acidic catalyst (one selected from HCl, benzenesulfonic acid, Amberlyst-15 acidic resin, and ZnCl2) accounting for 0.05-10% of the mass of fructose was added to the system. The reaction bottle was heated in an oil bath at 120° C. with a stirring speed of 800 rpm. After the reaction was completed, the reaction mixture was immediately cooled to room temperature and allowed to stand at room temperature for 2 hours, during which betaine hydrochloride crystallized and precipitated out. The solid was separated, and the acetone organic phase was collected separately. After dilution with deionized water, the fructose conversion rate and HMF yield were determined by HPLC. It can be observed that the introduction of an acidic catalyst can accelerate the reaction rate (see in Table 6).

TABLE 6
Effect of Acidic Catalysts on the Conversion of Carbohydrates
to HMF in the Acetone/Betaine Hydrochloride Solvent System

| | | | | Fructose | |
|---|---|---|---|---|---|
| | | Catalyst | | Con- | |
| | | Dosage | Reaction | version | HMF |
| Sample | | (wt % relative | Time | Rate | Yield |
| No. | Catalyst | to fructose) | (min) | (%) | (%) |
| 1 | HCl | 5 | 15 min | 100 | 88.32 |
| 2 | Benzene- | 3 | 20 min | 100 | 90.21 |
| | sulfonic | | | | |
| | acid | | | | |
| 3 | Amberlyst-15 | 5 | 20 min | 100 | 89.76 |
| | acidic resin | | | | |
| 4 | ZnCl₂ | 5 | 20 min | 87.25 | 88.62 |

What is claimed is:

1. A method for preparing 5-hydroxymethylfurfural in a temperature-controlled phase separation solvent system, comprising:
S1: Constructing a temperature-controlled phase separation solvent system; wherein the solvent system comprises an aqueous betaine hydrochloride solution and an organic solvent, the organic solvent is acetone; the volume ratio of the organic solvent to the aqueous betaine hydrochloride solution ranges from 1:1 to 5:1, and the concentration of betaine hydrochloride in the aqueous betaine hydrochloride solution ranges from 25 to 40 wt %;
S2: Adding a defined amount of carbohydrate to the solvent system and heating the mixture to conduct a dehydration reaction, with the aqueous betaine hydrochloride solution serving as the reaction phase and the organic solvent serving as the extraction phase to form phase stratification; wherein the reaction temperature ranges from 100 to 180° C., the reaction time ranges from 30 to 180 min, and the concentration of the carbohydrate ranges from 5 to 40 wt %;
S3: After completion of the reaction, cooling the reaction mixture at room temperature for a period of time, whereby betaine hydrochloride precipitates in crystalline form; the product is present in a mixed solution of the organic phase and deionized water, and 5-hydroxymethylfurfural is obtained via isolation and purification of the mixed solution;
wherein in step S2, the carbohydrate is selected from any one of fructose, high-fructose syrup, and inulin.

2. The method according to claim 1, comprising the steps of:
immediately cooling the reaction mixture to room temperature and standing at room temperature for a period of time until most of the betaine hydrochloride crystallizes and precipitates out;
separating and collecting the betaine hydrochloride and the organic phase respectively;
distilling the organic phase to obtain the 5-hydroxymethylfurfural; and the recovered organic solvent and betaine hydrochloride are directly recycled for subsequent use.
